(19) United States
(12) Patent Application Publication
(10) Pub. No.: US 2010/0217093 A1
(43) Pub. Date: Aug. 26, 2010

(54) PORTABLE BIOLOGICAL INFORMATION MEASURING DEVICE

(75) Inventors: Li Li Ko, Taipei (TW); Hsin Yi Pai, Sanchong City (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: MICRO-STAR INTERNATIONA'L CO., LTD., Taipei County (TW)

(21) Appl. No.: 12/545,396

(22) Filed: Aug. 21, 2009

(30) Foreign Application Priority Data
Feb. 25, 2009 (TW) 098106052

(51) Int. Cl. A61B 5/00

(52) U.S. Cl. 600/300

(57) ABSTRACT

A portable biological information measuring device includes a body and at least one measuring member. The body has a biological information measuring circuit assembly and a hub. The measuring member has a connection line, which coils in the hub and is capable of changing a pull-out length according to a disposition position of the body on a human body. In this manner, the body is disposed at different positions according to measuring demands of users, such that the connection line may freely extend to the appropriate length for the user, for performing a measurement.

### CROSS-REFERENCE TO RELATED APPLICATIONS

This non-provisional application claims priority under 35 U.S.C. §119(a) on Patent Application No(s). 098106052 filed in Taiwan, R.O.C. on Feb. 25, 2009, the entire contents of which are hereby incorporated by reference.

### BACKGROUND OF THE INVENTION

1. Field of Invention

The present invention relates to a biological information measuring device, and more particularly to a portable biological information measuring device.

2. Related Art

Along with the ever-increasing development of living standard and medical science, people attach great importance to health management, such that researches on various medical devices and sensing instruments are increasingly concerned. Therefore, various devices capable of measuring signals generated by the human body, for example, the medical level apparatus used in the hospitals or the household consumable instruments for health care, are proposed.

In various items of the human body checkup, electro-cardiogram measurement is a quite important checkup item. In general, the doctors performs analysis and diagnosis by using the electro-cardiograms generated by the electro-cardiograph, so as to diagnose the arrhythmia, the myocardial infarction, the angina pectoris, or other changes resulting from the heart diseases, such as bradycardia. In addition, the changes of the electrolytes in the human body, for example, calcium and potassium ions, may be reflected and observed from the electro-cardiogram.

The electro-cardiograph recently used in the medical institution has strong functions, and is capable of accurately measuring the required information according to various symptoms and the corresponding waveforms. The volume of the electro-cardiograph is quite large, so it is limited in using by reason of time and location. However, certain diseases (such as arrhythmia or coronary insufficiency) usually occur unpredictably at any time. Therefore, for certain patients, the change of the electro-cardiogram cannot be immediately monitored, such that it is quite dangerous and inconvenient.

Accordingly, a modified electro-cardiograph is developed on the market, and the electro-cardiograph having a small volume is portable, and even may be combined with the clothes to wear the electro-cardiograph directly on the human body. Therefore, during measurement, the measurement may be performed as long as the electrode patches connected to the electro-cardiograph are adhered to the user body. In this manner, the user may perform the measurement according to the using occasions, so it is flexible and convenient in using, and the user may know the change of the human body according to the data obtained by measurement.

However, the existing electro-cardiograph still has quite a lot of inconveniences in operation and using. For example, the electrode patches in the electro-cardiograph are mostly connected to the electro-cardiograph through a plurality of wires, and the length of the wire is relatively redundant on design, so as to result in the inconvenience in accommodation. The excessively redundant wires are easily winded and knotted during the using process, it takes time for the user to unwind the winded wires, and even the winded and knotted wires may result in break of the signal lines, thereby affecting the using life. In addition, when the user intends to adhere the electrode patches to certain members of the human body for performing the measurement, the length of the wires connected to the electrode patches are fixed, such that the user cannot make the wires freely extend to the appropriate length according to the positions to be measured, so the user feels quite embarrassed and inconvenient. Further, the common portable electro-cardiograph on the market is directly worn on the waist of the user. However, when the user performs the relatively strenuous exercises, the electro-cardiograph worn on the waist easily falls on the ground, and is damaged.

### SUMMARY OF THE INVENTION

In view of the above problems, the present invention is a portable biological information measuring device, which is convenient for the user to perform a measurement, thereby solving the problem of the conventional electro-cardiograph that it is extremely inconvenient in operation and using.

The portable biological information measuring device according to the present invention comprises a body and at least one measuring member. The body has a biological information measuring circuit assembly and a hub disposed therein. The measuring member has a connection line, which coils in the hub and is capable of changing a pull-out length according to a disposition position of the body on a human body.

In the portable biological information measuring device according to an embodiment of the present invention, if the body of the portable biological information measuring device is hung on the chest, the user may pull out the connection line to a desired length for performing the measurement according to a distance between the body and the chest and according to scale marks on the connection line. If the user intends to sleeve the body on the arm, the user may pull out the connection line to the desired length for performing the measurement according to a distance between the body and the arm and according to the scale marks on the connection line. If the user intends to clip the body on the neck, the user may pull out the connection line to the desired length for performing the measurement according to a distance between the body and one end of the neck and according to the scale marks on the connection line.

Therefore, in the portable biological information measuring device according to the present invention, the connection line coils in the hub disposed in the body, such that the pull-out length of the connection line may be changed according to a disposition position of the body on the human body. In this manner, the user may freely make the connection line extend to the desired position for measuring, and then performs the measurement through the measuring member. In addition, according to the demands of different positions for measuring, the user may directly pull out the connection line to the desired length for performing the measurement according to the scales on the connection line. Further, through the design of a stopper, the connection line can be fixed in different stages, thereby achieving a multi-stage positioning function. As compared with the conventional art, the portable biological information measuring device according to the present invention has a better convenience in using.

### BRIEF DESCRIPTION OF THE DRAWINGS

The present invention will become more fully understood from the detailed description given herein below for illustration only, and thus are not limitative of the present invention, and wherein:

FIG. 1 is a schematic view of a portable biological information measuring device according to an embodiment of the present invention;

FIG. 2 is a schematic three-dimensional exploded view of the portable biological information measuring device according to an embodiment of the present invention; and

FIGS. 3 to 7 are schematic views of different implementing aspects of the portable biological information measuring device according to an embodiment of the present invention.

### DETAILED DESCRIPTION OF THE INVENTION

A portable biological information measuring device according to the present invention may be, but not limited to, an electro-cardiograph, a pulsimeter, an oximeter, an electromyography (EMG), or other measuring devices. In the detailed description of the present invention as follows, the electro-cardiograph is used as a preferred embodiment of the present invention. However, the accompanying drawings are only provided for reference and description without limiting the protection scope of the present invention.

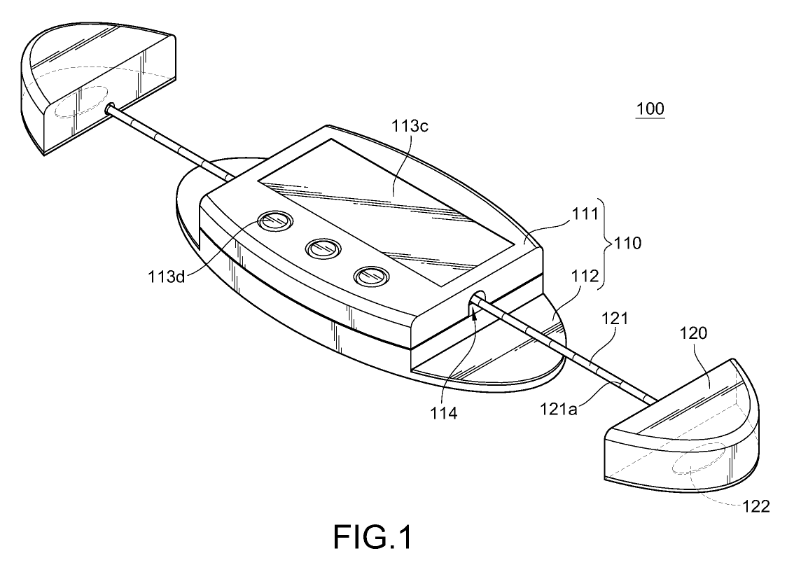

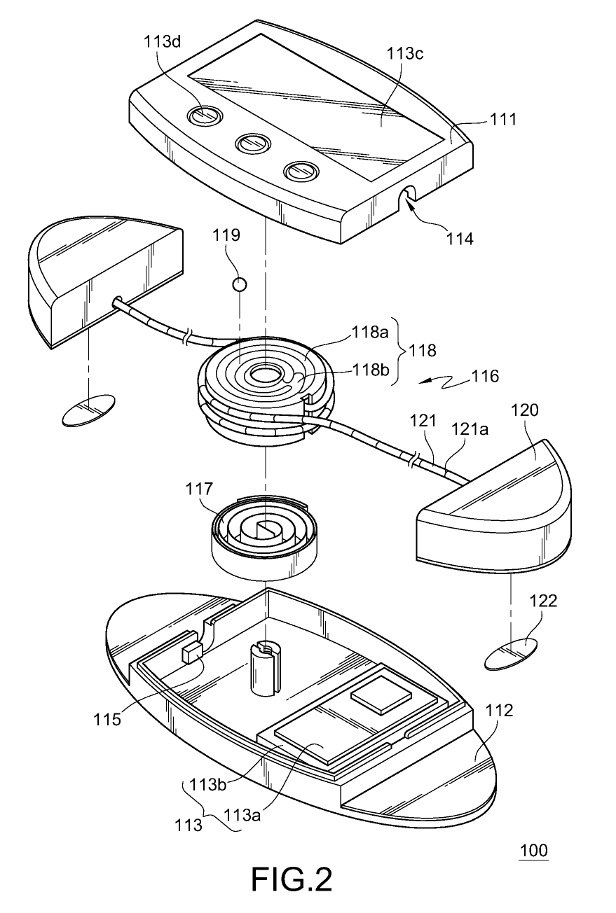

FIG. 1 is a schematic view of a portable biological information measuring device according to an embodiment of the present invention, and FIG. 2 is a schematic three-dimensional exploded view of the portable biological information measuring device according to an embodiment of the present invention. Referring to FIGS. 1 and 2, a portable biological information measuring device 100 comprises a body 110, a biological information measuring circuit assembly 113 disposed in the body 110, and two measuring members 120. The body 110 is composed of an upper case 111 and a lower case 112, and the two measuring members 120 are respectively electrically connected to the biological information measuring circuit assembly 113 through a connection line 121. A hub 116 is disposed in the body 110, such that the connection line 121 coils in the hub 116. In this embodiment, the measuring members 120 are disposed on two opposite sides of the body 110. The biological information measuring circuit assembly 113 disposed in the body 110 comprises, for example, a circuit board 113a, a processing chip (not shown), a cell 113b, a flash memory (not shown in the FIGS. 1 and 2), and other electronic elements. The biological information measuring circuit assembly 113 further comprises a liquid crystal display 113c, keys 113d, and other operation display elements exposed on an outer side of the body 110, which are well known by those of ordinary skill in the art, so they are not described and limited herein.

Particularly, the connection line 121 coils in the hub 116, and a pull-out length of the connection line 121 may be changed according to a disposition position of the body 110 on the human body. In this embodiment, the measuring member 120 is made of a conductive plastic material. However, in another embodiment of the present invention, the measuring member 120 may also be made of a conductive metal material, which is not limited thereto.

Referring to FIGS. 1 and 2, a slot 114 is defined in a side wall of the body 110 for passing through the connection line 121. At least one micro-switch 115 is further disposed on an inner side of the body 110 adjacent to the slot 114, such that the connection line 121 contacts with the micro-switch 115 along an accommodation moving direction or extension moving direction, thereby generating an off action or on action. More specifically, the micro-switch 115 is adjacent to the slot 114, for detecting a signal of a coiling action of the connection line 121, and delivering the detected signal to the circuit board 113a, such that the portable biological information measuring device 100 is turned on or off. In other words, when the user pulls the connection line 121 connected to the measuring member 120 out of the body 110 away from the slot 114, the portable biological information measuring device 100 is automatically turned on. On the contrary, when the user accommodates and makes the connection line 121 coil in the body 110, the portable biological information measuring device 100 is automatically turned off. In this manner, the portable biological information measuring device 100 can be automatically turned on or off according to the different actions of connection line 121 detected by the micro-switch 115, thereby effectively reducing the cost of the power source, and having the preferred convenience in using.

Referring to FIGS. 1 to 2, the hub 116 further comprises a coil spring 117 and a rotary disc 118, in which the coil spring 117 and the rotary disc 118 are pivoted in the body 110. The connection line 121 passes through the rotary disc 118 and is winded on the rotary disc 118, and two ends of the connection line 121 may respectively pass through the slots 114 on the two ends of the body 110. The coil spring 117 coils to a form volute shape, and the coil spring 117 may be accommodated and fixed in the rotary disc 118 after coiling, such that the coil spring 117 constantly applies a coiling force to the rotary disc 118 and the connection line 121. Particularly, the hub 116 further comprises a stopper 119 disposed in the hub 116. A guide slot 118a and a limit slot 118b are annularly disposed on a lateral side of the rotary disc 118. The guide slot 118a and the limit slot 118b are adjacent to and communicate with each other. In this embodiment, the stopper 119 is a ball, so when the rotary disc 118 rotates, the ball is disposed in the corresponding guide slot 118a on the rotary disc 118 in a rolling manner, and the ball is correspondingly limited in the limit slot 118b communicating with the guide slot 118a, such that the ball is positioned and the connection line 121 is fixed. It should be noted that the connection line 121 of this embodiment may be winded on the rotary disc 118, and the two ends thereof respectively pass through the body 110 from the slots 114. However, in another embodiment, one end of the connection line 121 is fixed on the rotary disc 118, and the other end thereof passes through the body from the slot 114 according to the demands. In addition, the stopper 119 of this embodiment is, for example, but is not limited to a ball. In another embodiment according to the present invention, the stopper 119 may also be a stopping piece.

It should be noted that the connection line 121 may be capable of achieving the multi-stage positioning function through the stopper 119 in the hub. Particularly, when the connection line 121 is pulled out for a pull-out length, the stopper 119 may generate a stopping action, such that the connection line 121 can be fixed on the determined length. If the user intends to release the stopping action, the connection line 121 is further pulled out for another length, and thus the coiling force is applied to the rotary disc 118 and the connection line 121 by the coil spring 117, thereby releasing the stopping action automatically, so as to automatically coil the connection line 121 back. Therefore, the connection line 121 is capable of achieving the multi-stage positioning function.

On the other hand, in order to enable the user to clearly and conveniently pull out the connection line to the required pull-out length, a plurality of scales 121a may be marked on the connection line 121. Particularly, according the user demands for measurement and according to the scales 121a on the connection line 121, the connection line 121 is pulled out to a specific length. When the user pulls out the connection line 121 to a pull-out length, here, the stopper 119 in the hub 116 rolls in the guide slot 118a and is limited in the limit slot 118b. At this situation, the connection line 121 is thus positioned on the specific scales 121a, and the pull-out length may be clearly shown. In practical using, as the connection line 121 may coil and be accommodated in the hub 116, the user may freely make the connection line 121 extend to the appropriate length for the human body for performing the measurement according different wearing modes.

In addition, the measuring member 120 further has a conductive patch 122, for example, a conductive silica gel film, capable of contacting with and being adhered to the human body, for sensing electrocardiogram signals of the human body. Particularly, when the user adheres the measuring member 120 to the measured position of the human body, the conductive patch 122 contacts with the skin of the user, so as to sense the electrocardiogram signals, the sensed electrocardiogram signals are transmitted to the biological information measuring circuit assembly 113 in the body 110 through the connection line 121, and then the received electrocardiogram signals are stored, analyzed, and displayed by the liquid crystal display LCD 113c.

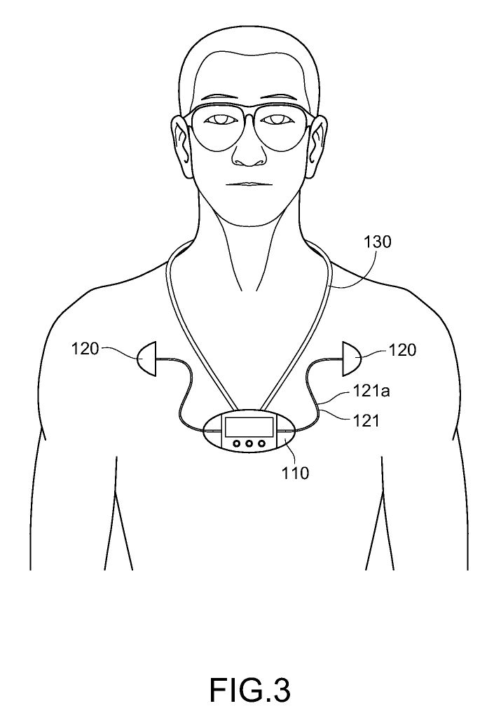

According to an embodiment of the present invention, different wearing modes of the portable biological information measuring device are illustrated as follows. FIGS. 3 to 7 are schematic views of the different wearing modes of the portable biological information measuring device according to an embodiment of the present invention. Referring to FIG. 3, the portable biological information measuring device 100 may be sleeved on the human body through an elastic fixing member 130. Particularly, the elastic fixing member 130 extends outwards from one end of the body 110, such that the body 110 is elastically sleeved on the human body. In this embodiment, the elastic fixing member 130 is a chain.

Referring to FIG. 3, if the left and the right sides of the chest are the positions to be measured, the body 110 is hung on the user's chest through the elastic fixing member 130. At this situation, the required measured length is a distance between the body 110 and the left side or the right side of the chest, for example, 10 cm. Thus, the only thing that the user needs to do is to pull out the connection line 121 to a length of 10 cm for performing the measurement according to the scales 121a marked on the connection line 121. In other words, the pull-out length of the connection line 121 is the distance between the position of the body 110 and the position to be measured, such that the pull-out length of the connection line 121 through the scales 121a on the connection line 121 can be clearly shown. However, the connection line 121 can be slightly pulled by the user to coil the pulled out connection line 121 after finishing the measurement. At this situation, the connection line 121 coils and is accommodated in the hub 116 through the automatic coiling force between the rotary disc 118 and the coil spring 117.

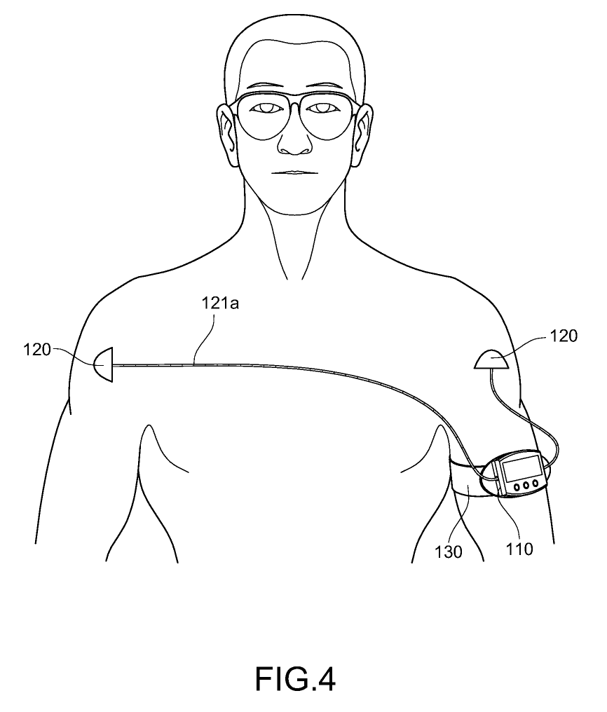

Referring to FIG. 4, the body 110 is surrounded on the arm through the elastic fixing member 130 if the user intends to put the body 110 on the arm. In this embodiment, the elastic fixing member 130 further comprises a hook and loop fastener, for fixing the body 110 on the arm. Particularly, if the two arms are the positions to be measured, the body 110 is sleeved on one of the user's arms through the elastic fixing member 13. At this situation, the required measured length is a distance between the body 110 and the left and the right arms, for example, respectively 10 cm and 30 cm, and therefore, the user can pull out one end of the connection line 121 to a length of 10 cm and pull out the other end to a length of 30 cm for performing the measurement according to the scales 121a marked on the connection line 121. In other words, the pull-out length of the connection line 121 is the distance between the position of the body 110 and the position to be measured, such that the pull-out length can be clearly shown through the scales 121a on the connection line 121.

In addition, the body 110 may be conveniently and stably fixed on the user's arm through the elastic fixing member 130. The body 110 affixed on the arm may not fall down while the user uses the portable biological information measuring device 100 during exercise, so it is conveniently carried, and the damage of the portable biological information measuring device 100 is reduced

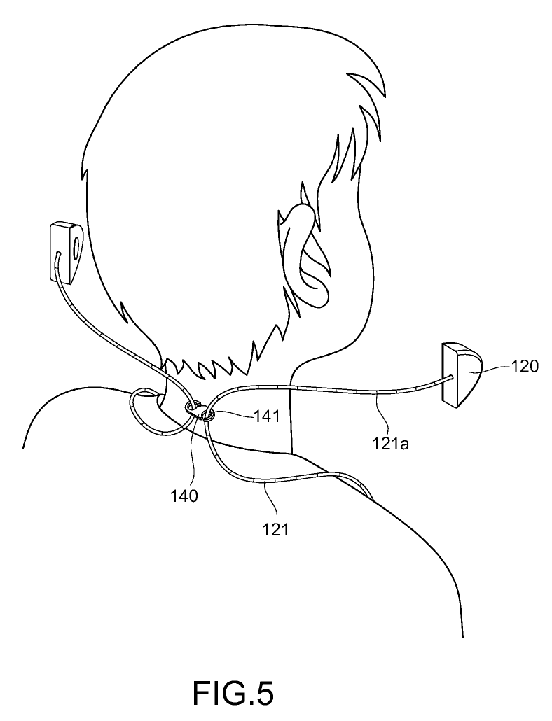

Referring to FIG. 5, the body 110 may be directly worn on the human body without additionally disposing the elastic fixing member 130, thereby improving the convenience in use. Referring to FIG. 5, the portable biological information measuring device 100 further comprises a sliding member 140, and the sliding member 140 is movably sleeved on the connection line 121. The sliding member 140 has two perforations 141, providing for the connection line 121 to pass through. The materials of the outer surface of the connection line 121 can be rubber, plastic, or other flexible materials according to the demands, and therefore the connection line 121 may pass a notch defined in the sliding member 140 in a deformed state, such that the connection line 121 passes through the perforation 141 or is taken out of the perforation 141. Further, the connection line 121 is limited in the perforation 141 through the sliding member 140, and the connection line 121 may be pulled to slide in the perforation 141 without being taken out of the perforation 141. Therefore, the user pulls out the connection line for a specific length, and winds the connection line 121 to an appropriate sleeve hole for the user's neck, thus facilitating the wearing for the user. Then, the sliding member 140 limits the connection line 121 from moving, thereby directly performing the measurement through the measuring member 120. In this manner, the connection line 121 surrounds the neck through the disposition of the sliding member 140, thereby omitting the fixing member additionally disposed on the body 110. Next, through the design of the sliding member 140, the excessively redundant connection line 121 is prevented from being winded and knotted during the using process, thereby enhancing the using life of the connection line 121. In addition, the sliding member 140 of this embodiment is disposed on the back of the neck after the connection line 121 is winded, or may be hung on the chest after the connection line 121 is winded, which depend on the convenience of the user in use, and are not limited here.

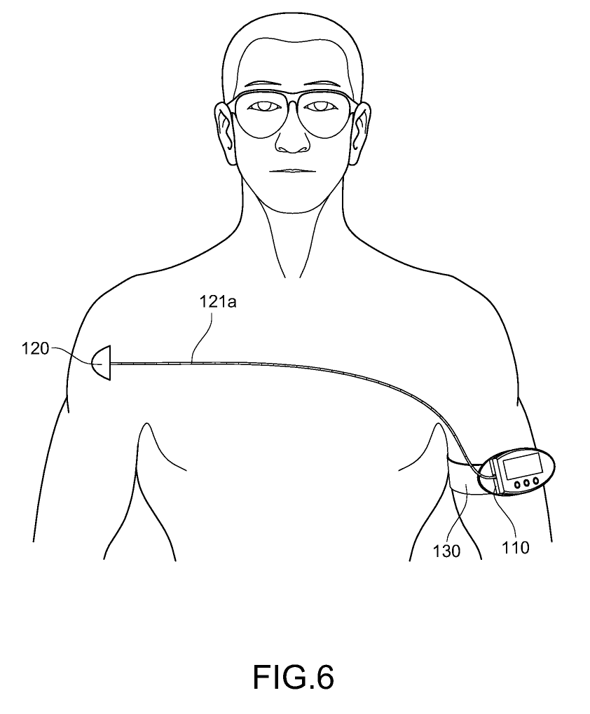

Referring to FIG. 6, for the two measuring members of this embodiment, one measuring member 120 is connected to the connection line 121, and the other measuring member (not shown) is disposed on an outer lateral side of the lower case 112, that is, is disposed on a lateral side of the body 110 directly contacting with the human body. In other words, one measuring member (not shown) is fixed on the body 110, and the other measuring member 120 is connected to the connection line 121 and may be pulled out. Thus, only the pull-out length of one end of the connection line 121 is necessary to be controlled for performing the measurement.

Particularly, if the two arms are the positions to be measured, the body 110 is sleeved on one user's arm through the elastic fixing member 13. At this situation, the required measured length is the distance between the body 110 and the left and the right arms, for example, respectively 40 cm. Thus, the only thing that the user needs to do is to pull out the connection line 121 connected to the measuring member 120 to a length of 40 cm for performing the measurement according to the scales 121a marked on the connection line 121. In other words, the pull-out length of the connection line 121 is the distance between the position of the body 110 and the position to be measured, such that the pull-out length through the scales 121a on the connection line 121 can be clearly shown.

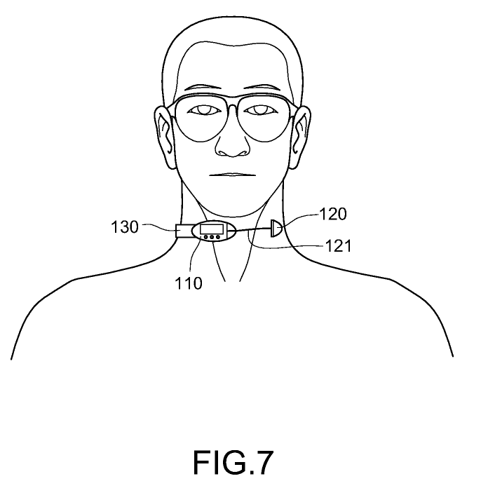

Referring to FIG. 7, the portable biological information measuring device 100 can be also disposed on the neck to directly measure the information according to the demands. That is to say, the body 110 is worn on the neck in the surrounding manner through the elastic fixing member 130 of this embodiment, for example, an annular member. Therefore, when the user surrounds the elastic fixing member 130 on the neck, the body 110 may be stably fixed on the neck. In this manner, the measuring member 120 connected to the connection line 121 may be pulled out to a pull-out length for performing the measurement, and the other end performs the measurement through the measuring member (not shown) disposed on a lateral side of the body 110 directly contacting with the neck. Particularly, if the neck is the position to be measured, the body 110 is clipped on the neck of the user by the elastic fixing member 130. At this situation, the required measured length is the distance between the body 110 and one end of the neck, for example, 30 cm. Thus, the only thing that the user needs to do is to pull out the connection line 121 connected to the measuring member 120 to a length of 30 cm for performing the measurement according to the scales 121a marked on the connection line 121. In other words, the pull-out length of the connection line 121 is the distance between the position of the body 110 and the position to be measured, such that the pull-out length through the scales 121a on the connection line 121 can be clearly shown.

What is claimed is:

1. A portable biological information measuring device, comprising:
a body, having a biological information measuring circuit assembly and a hub disposed therein; and
at least one measuring member, having a connection line, wherein the connection line coils in the hub, and is capable of changing a pull-out length according to a disposition position of the body on a human body.

2. The portable biological information measuring device according to claim 1, further comprising a stopper disposed in the hub, wherein the stopper is capable of a multi-stage positioning of the connection line.

3. The portable biological information measuring device according to claim 2, wherein the connection line has a plurality of scales, and is positioned at the scales through the stopper.

4. The portable biological information measuring device according to claim 1, further comprising a sliding member, wherein the sliding member is movably sleeved on the connection line, such that the connection line is winded to become a sleeve hole at an appropriate length, and the sliding member limits the connection line from moving.

5. The portable biological information measuring device according to claim 1, further comprising an elastic fixing member, extending outwards from one end of the body, such that the body is elastically sleeved on the human body.

6. The portable biological information measuring device according to claim 1, further comprising at least one micro-switch disposed on a side wall of the body, wherein the connection line contacts with the micro-switch along an accommodation moving direction or extension moving direction, thereby generating an off action or an on action.

7. The portable biological information measuring device according to claim 2, wherein the hub further comprises a coil spring and a rotary disc, the coil spring and the rotary disc are pivoted in the body, the connection line passes through the rotary disc and is winded on the rotary disc, and the rotary disc accommodates the coil spring, such that the coil spring constantly applies a coiling force to the rotary disc and the connection line.

8. The portable biological information measuring device according to claim 7, wherein the stopper is a ball disposed on a corresponding guide slot of the rotary disc in a rolling manner, and the ball is limited in a limit slot communicating with the guide slot to position the ball and fix the connection line.

9. The portable biological information measuring device according to claim 1, wherein the measuring member is made of a conductive metal material.

10. The portable biological information measuring device according to claim 1, wherein the measuring member is made of a conductive plastic material.

11. The portable biological information measuring device according to claim 2, wherein the stopper is a stopping piece.
